(19) United States
(12) Patent Application Publication
(10) Pub. No.: US 2013/0165801 A1
(43) Pub. Date: Jun. 27, 2013

(54) PASSIVE PRESSURE SENSOR FOR IMPLANTABLE LEAD

(71) Applicant: Xiaoyi Min, Thousand Oaks, CA (US)

(72) Inventor: Xiaoyi Min, Thousand Oaks, CA (US)

(73) Assignee: PACESETTER, INC., Sylmar, CA (US)

(21) Appl. No.: 13/333,734

(22) Filed: Dec. 21, 2011

(51) Int. Cl. A61B 5/0215; A61N 1/05

(52) U.S. Cl. 600/486; 607/119

(57) ABSTRACT

A passive pressure sensor is used with an implantable lead to measure pressure within a patient's heart. In some embodiments, the passive pressure sensor is incorporated into an implantable lead. In other embodiments, the passive pressure sensor is incorporated into a device that is slid onto an implantable lead.

### TECHNICAL FIELD

This application relates generally to pressure sensors and more specifically, but not exclusively, to a pressure sensor for an implantable lead.

### BACKGROUND

Heart failure is a debilitating disease in which abnormal function of a patient's heart leads to inadequate blood flow to the patient's body. While a heart failure patient may not suffer debilitating symptoms immediately, with few exceptions, the disease is relentlessly progressive. Moreover, as heart failure progresses, it may become increasingly difficult to manage.

Despite current drug and device therapies, the rate of heart failure hospitalization still remains high. Consequently, significant hospitalization costs are incurred annually for heart failure patients.

Pulmonary artery pressure is a known predictor for heart failure progression. Consequently, it has been proposed to place a dedicated pressure sensor in a branch of the pulmonary artery for heart failure monitoring. In practice, however, there are risks associated with the dedicated implant procedure used for such a sensor.

In addition, it has been proposed to incorporate active pressure sensors on implantable leads to measure ventricular pressure or atrial pressure. However, these types of sensors are generally quite complicated and have a relatively high cost.

Accordingly, a need exists for more effective techniques for monitoring heart failure status of patients so that appropriate treatment may be readily prescribed for the patients, thereby lowering the hospitalization rate for the patients.

### SUMMARY

A summary of several sample aspects of the disclosure and embodiments of an apparatus constructed or a method practiced according to the teaching herein follows. It should be appreciated that this summary is provided for the convenience of the reader and does not wholly define the breadth of the disclosure. For convenience, one or more aspects or embodiments of the disclosure may be referred to herein simply as “some aspects” or “some embodiments.”

The disclosure relates in some aspects to a passive pressure sensor that is used with an implantable lead (e.g., a standard right ventricle (RV) pacing/sensing lead or a high voltage lead). In some embodiments, the passive pressure sensor is incorporated into an implantable lead. In other embodiments, the passive pressure sensor is incorporated into a device that is slid onto an implantable lead.

The passive pressure sensor comprises a resonant inductor-capacitor circuit that is excited by an electromagnetic field generated by an external monitoring system. The capacitive circuit portion of the resonant circuit is flexible such that changes in pressure at the pressure sensor (e.g., implanted in a patient's heart) cause changes in the capacitance of the capacitive circuit. Thus, changes in pressure at the pressure sensor are reflected by changes in the resonant frequency of the excited resonant circuit. These changes in the resonant frequency are then detected by the external monitoring system. Accordingly, a passive pressure sensor as taught herein may be effectively employed to monitor and, therefore, treat heart failure (e.g., by monitoring changes in blood pressure that are indicative of heart failure).

For example, when incorporated with an RV lead, the passive pressure sensor may be used to measure RV pressure, dP/dt, and estimated pulmonary artery pressure. To this end, the passive pressure sensor may be located at various locations along the implantable lead, whereby the implantable lead is oriented upon implant to place the passive pressure sensor at a desired location within the heart.

There are several potential advantages over existing systems provided by a lead-based passive pressure sensor as taught herein. No changes are required for pacer hardware and firmware, since the pressure sensor may communicate directly through telemetry to an external monitoring system (e.g., which may be integrated with an implantable device programmer). No significant added surgical procedures or implant time is needed since the pressure sensor is implanted with or in conjunction with the implantation of the lead. There is less clinical risk since the pressure sensor is either fully integrated into or onto a standard lead. There is less clinical risk since the pressure sensor is not implanted in the pulmonary artery or across the intra-atrial septum. There is lower cost due to the use of low complexity circuits. Portability may be improved since a more efficient telemetry design that is integrated with the whole system of a programmer, a pacer/ICD/CRT, and a telemetry system may be employed.

### BRIEF DESCRIPTION OF THE DRAWINGS

These and other aspects of the disclosure will be more fully understood when considered with respect to the following detailed description, the appended claims, and the accompanying drawings, wherein:

FIG. 1 is a simplified diagram of a distal end of an implantable lead incorporating a passive pressure sensor;

FIG. 2 is a simplified diagram of view A of the implantable lead of FIG. 1;

FIG. 3 is a simplified diagram illustrating one embodiment of an inductive circuit;

FIG. 4 is a simplified diagram illustrating another embodiment of an inductive circuit;

FIG. 5 is a simplified diagram illustrating one embodiment of a capacitive circuit;

FIG. 6 is a simplified diagram illustrating another embodiment of a capacitive circuit;

FIG. 7 is a simplified diagram of a side view of a device incorporating a passive pressure sensor;

FIG. 8 is a simplified diagram of a perspective view of a device incorporating a passive pressure sensor;

FIG. 9 is a simplified diagram illustrating an example of a procedure for installing a passive pressure sensor device onto an implantable lead;

FIG. 10A is a simplified diagram illustrating an example of a passive pressure sensor device incorporated onto an implantable lead;

FIG. 10B is a simplified diagram illustrating another example of a passive pressure sensor device incorporated onto an implantable lead;

FIG. 11 is a simplified diagram illustrating an example of another procedure for installing a passive pressure sensor device onto an implantable lead;

FIG. 12 is a simplified diagram illustrating another example of a passive pressure sensor device incorporated onto an implantable lead;

FIG. 13 is a simplified diagram of an embodiment of an implantable stimulation device in electrical communication with one or more leads implanted in a patient's heart for sensing conditions in the patient, delivering therapy to the patient, or providing some combination thereof;

FIG. 14 is a flowchart of sample operations that may be performed to implant a passive pressure sensor device.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

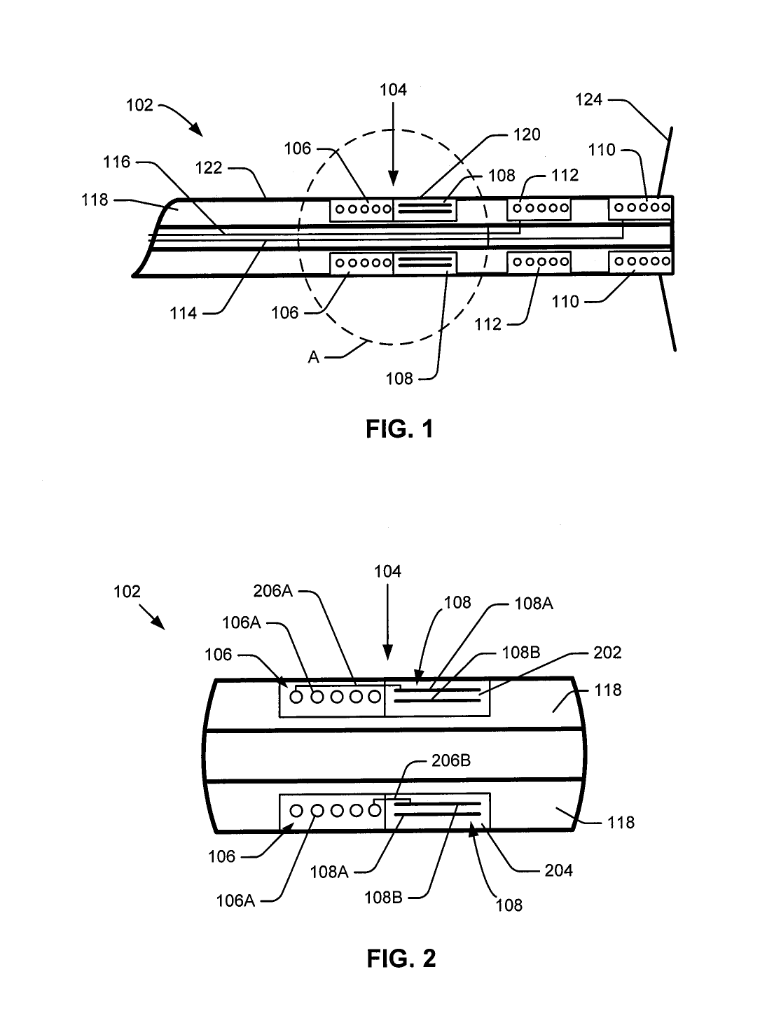

FIG. 1 illustrates, in a simplified sectional side view, an embodiment of an implantable lead 102 that incorporates a passive pressure sensor 104 in accordance with the teaching herein. The pressure sensor 104 comprises an inductive circuit 106 (e.g., a wound inductor) and a capacitive circuit 108 (e.g., a pair of conductive plates separated by a dielectric material).

In this example, the pressure sensor 104 is incorporated into a distal section (e.g., the header) of the lead 102. It should be appreciated, however, that the pressure sensor 104 may be incorporated into different locations along the length of the lead to facilitate obtaining pressure measurements from different areas of the heart.

The pressure sensor 104 is electrically isolated from all other electrical circuits of the lead 102. For example, the lead 102 includes a tip electrode coil 110 and a ring electrode coil 112 that are coupled to conductors 114 and 116, respectively. However, the electrical conductors of the inductive circuit 106 and the capacitive circuit 108 are insulated from the conductors 114 and 116 and the coils 110 and 112 (e.g., via insulation material on the conductive materials and/or a gap in the interior of the lead 102).

FIG. 1 also illustrates that in some embodiments the pressure sensor 104 is located adjacent an exterior surface of the biocompatible lead body 118 of the lead 102. In this example, an exterior surface 120 of the pressure sensor 104 is coplanar with the exterior surface 122 of the lead body 118. Thus, the external surface 120 of the pressure sensor would be flexible (e.g., to couple pressure waves to the capacitive circuit 108) and biocompatible in this case. For example, the external surface may comprise silicone or some other flexible biocompatible material. In other embodiments, however, the pressure sensor 104 may be located completely within the lead body 118. In these cases, the pressure sensor need not be biocompatible.

In the example of FIG. 1, the lead 102 is shown as including a passive fixation element 124. It should be appreciated, however, that a passive pressure sensor as taught herein may be incorporated into an implantable lead employing active fixation or into some other type of implantable lead.

FIG. 2 is an enlarged representation of the view A of FIG. 1. This figure illustrates the connectivity and structure of the pressure sensor 104 in more detail. In particular, in conjunction with FIGS. 3-6, FIG. 2 serves to illustrate that the pressure sensor 104 may take the form of an inductive-capacitive (LC) resonant circuit having a cylindrical structure.

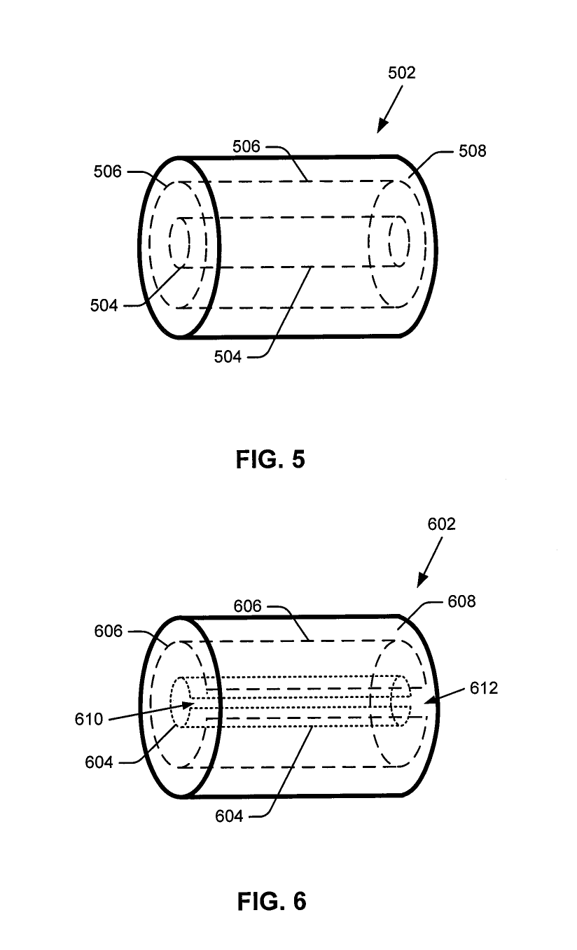

As represented by the plates 108A and 1088 of the capacitive circuit 108 in FIG. 2 (and as further represented in FIGS. 5 and 6), each plate of the capacitive circuit 108 may take the form of a cylinder or a partial cylinder. Here, each cylinder is oriented in a longitudinal direction along the longitudinal axis of the lead body 118. That is the longitudinal axis of each cylinder is parallel with (or, in some cases, the same as) longitudinal axis of the lead body 118. Due to this large plate surface area that this configuration provides, the plates 108A and 108B of the capacitive circuit 108 may be more susceptible to relative deformation when the lead 102 is subjected to changes in external pressure. Consequently, the resonant circuit comprised of the capacitive circuit 108 and the inductive circuit 106 will be more sensitive to pressure changes, thereby facilitating more accurate pressure readings in some cases.

FIG. 2 also illustrates that a relatively flexible dielectric material 202 (e.g., a fiberglass material) may be disposed between the plates 108A and 108B of the capacitive circuit 108. In this way, external pressure induced on the lead 102 may more easily cause the distance between the plates 108A and 108B to change. Thus, the resonant circuit comprised of the capacitive circuit 108 and the inductive circuit 106 will be more sensitive to pressure changes, thereby facilitating more accurate pressure readings in some cases.

A relatively flexible material 204 (e.g., a silicone-based material) may be disposed adjacent (e.g., next to or under) an exterior surface of the lead body 118 and engaged with (e.g., disposed against, in contact with, etc.) the capacitive circuit 108. The flexible material 204 may thus serve to couple pressure waves to the capacitive circuit 108 in an efficient manner. As discussed above, in some embodiments (e.g., as shown in FIG. 2), the flexible material 204 may comprise a portion of the outer surface of the lead. In this case, the flexible material 204 itself will form part of the hermetic seal for the lead 102, along with hermetic sealing (e.g., via adhesive or welding) between the flexible material 204 and lead body 118. For example, a thin layer of fiberglass (or some other suitable material) may be provided over an outer enclosure of the capacitive circuit 108 (or directly over an outer plate of the capacitive circuit 108).

In other embodiments (not shown in FIG. 2), the flexible material 204 may be housed entirely within (but located adjacent to) the lead body 118. In such a case, the biocompatible lead body 118 may provide the hermitic seal. In addition, the lead body 118 will be sufficiently flexible here to couple pressure waves to the capacitive circuit 108 (e.g., via the flexible material 204). For example, the lead body 118 may comprise a relatively thin outer layer (e.g., constructed of silicone, fiberglass, or some other suitable material) that covers an outer enclosure of the capacitive circuit 108 (or covers an outer plate of the capacitive circuit 108).

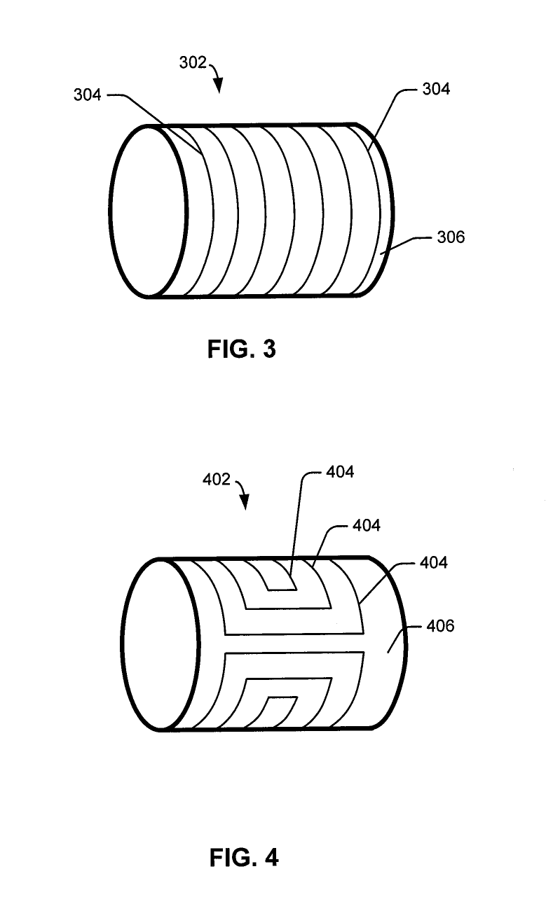

As represented by the conductor 106A of the inductive circuit 106 in FIG. 2 (and as further represented in FIGS. 3 and 4), inductive circuit 106 may take the form of a cylindrical coil or some other coil-like structure. For example, the coil conductor may start at the upper left circle of FIG. 2 (connected to a conductor 206A) and wrap around the interior of the lead 102, terminating at the lower right circle of FIG. 2 (connected to a conductor 206B).

The inductive circuit 106 may be constructed in various ways. In some embodiments, the inductive circuit 106 is constructed on a PEEK bobbin with DFT wire (41% AG or less) or copper wire. The wire may be coated with, for example, ETFE or some other insulation material. In some embodiments, the wire may be relatively thin (e.g., 100 micrometers to 2 mils) so that the coil may have large number of turns, thereby providing a higher value of inductance for a given size coil.

FIGS. 1 and 2 illustrate an embodiment where the inductive circuit 106 and the capacitive circuit 108 are physically located in a series relationship with respect to one another (i.e., one circuit is positioned further down the lead body 118 from the other circuit). In other embodiments (not shown), the capacitive circuit 108 may be located over the inductive circuit 106. That is, the inductive circuit 106 and the capacitive circuit 108 may have a concentric relationship with one another.

As FIG. 2 illustrates, one terminal of the inductive circuit 106 is coupled via the conductor 206A to the plate 108A of the capacitive circuit 208, while the other terminal of the inductive circuit 106 is coupled via the conductor 206B to the plate 108B of the capacitive circuit 108. Thus, the inductive circuit 106 and the capacitive circuit 108 are coupled in parallel, thereby forming a passive resonant circuit that is capable of being excited by an externally applied electromagnetic field.

The physical properties of the inductive circuit 106 (e.g., the number of turns) and the capacitive circuit 108 (e.g., size and distance between plates) are selected to provide a desired resonant frequency for the sensor circuit 104. In some embodiments, the resonant circuit has a resonant frequency of less 35 MHz or less (e.g., 30 MHz). Such a circuit may be compatible with other types of passive pressure sensors.

In some embodiments, the resonant circuit has a resonant frequency of 20 MHz or less (e.g., 10-15 MHz). This lower resonant frequency may be achieved, for example, as a result of the physical characteristics (e.g., the size and shape) of the passive pressure sensor that can be achieved in an implantable device based on the teachings herein. Such a circuit may advantageously enable the use of a smaller transmission coil at the external monitoring system or other similar device. Consequently, a more portable external monitoring system (or other device) may be employed to acquire pressure readings from a passive pressure sensor constructed in accordance with the teachings herein. Alternative, this smaller size may enable the transmission coil to be incorporated into an external device (e.g., a programmer) used for communicating with an implantable medical device (e.g., a pacemaker, an ICD, etc.).

FIGS. 3 and 4 illustrate sample inductive circuits that may be employed in the various embodiments described herein. In FIG. 3, an inductive circuit 302 takes the form of a coil of wire 304 that is wrapped around a cylindrical body 306. In FIG. 4, an inductive circuit 402 takes the form of a coil of wire 404 that is wrapped around a cylindrical body 406 in a more elaborate (e.g., looped-back) manner.

FIGS. 5 and 6 illustrate sample capacitive circuits that may be employed in the various embodiments described herein. In FIG. 5, a capacitive circuit 502 takes the form of a plurality (2 in this example) of concentric cylindrical plates. Here, an inner plate 504 lies within an outer plate 506. Typically, the plates 504 and 506 are embedded in a flexible material (or materials) 508 to provide the variable capacitance that is desired for the pressure sensor.

In practice, the configuration of FIG. 5 may not provide the desired degree of variable capacitance since the outer cylindrical plate may not be sufficiently compressible. A higher degree of variable capacitance may be provided by the configuration of FIG. 6.

In FIG. 6, a capacitive circuit 602 takes the form of a plurality (2 in this example) of concentric partially-cylindrical plates (e.g., substantially cylindrical plates). Again, an inner plate 604 lies within an outer plate 606. Also, the plates 604 and 606 are typically embedded in a flexible material (or materials) 608 to provide the variable capacitance that is desired for the pressure sensor.

Due to the presence of the gaps 610 and 612 between the ends of the plates 604 and 606, respectively, when subjected to external pressure, the edges of each plate 604 and 606 and may more easily move relative to one another. That is, the gaps 610 and 612 may become smaller and larger with changes in pressure. As a result, there will be more relative movement between the plates 604 and 606 (e.g., as manifested by a larger variance in the spacing between the plates 604 and 606), resulting in a larger change in the capacitance of the capacitive circuit 602. Hence, the configuration of FIG. 6 may be used to provide a more sensitive pressure sensor.

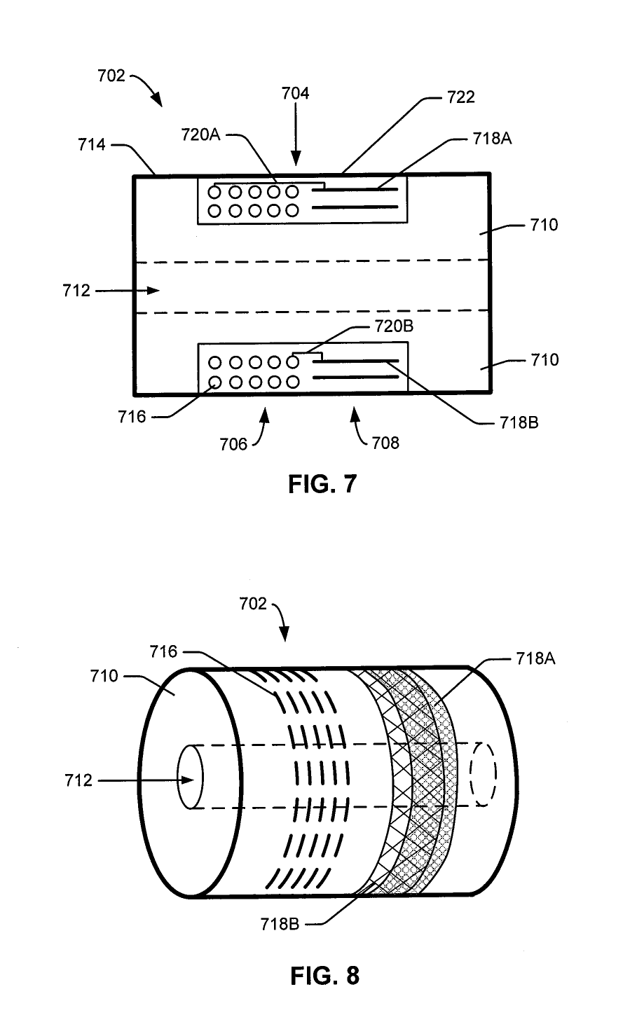

FIGS. 7 and 8 illustrate alternative embodiments of passive pressure sensor implemented as a cylindrical device (e.g., a ring structure) that may be installed on an implantable lead. FIG. 7 illustrates, in a simplified sectional side view, an embodiment of a pressure sensor device 702 that incorporates a passive pressure sensor 704 in accordance with the teaching herein. The pressure sensor 704 comprises an inductive circuit 706 (e.g., a wound inductor) and a capacitive circuit 708 (e.g., a pair of conductive plates separated by a dielectric material).

The pressure sensor device 702 comprises a biocompatible housing 710 (e.g., constructed of silicone, Optim, or some other suitable material) that defines a central hole 712 along the longitudinal axis of the housing 710. The inside diameter of the hole 712 may be sized slightly larger than the outside diameter of a lead body (e.g., between 1.5-2.5 millimeters, inclusive) to facilitate assembling the device 702 onto an implantable lead. In some embodiments, the inside diameter of the hole 712 may be sized smaller than the outside diameter of lead header (e.g. as shown in FIG. 12) to prevent the device 702 from sliding over the lead header.

The outside diameter and the length of the housing 710 are sized to facilitate implant via a transvenous approach. For example, in some embodiments, the outside diameter of the housing 710 may be 3 millimeters or less. In some embodiments, the housing 710 has an outer diameter of greater than 2 millimeters (e.g., wider than an implantable lead). Also, in some embodiments, the length of the housing 710 may 80 millimeters or less (e.g., to maintain a sufficient bend angle for an implantable lead).

FIG. 7 also illustrates that in some embodiments the pressure sensor 704 is located adjacent (e.g., next to or under) an exterior surface 714 of the housing 710. In this example, an exterior surface 722 of the pressure sensor 704 is coplanar with the exterior surface 714 of the housing. Thus, the external surface 722 of the pressure sensor 702 will be flexible (e.g., to couple pressure waves to the capacitive circuit 708) and biocompatible in this case. For example, the external surface 722 may comprise silicone or some other flexible biocompatible material. In addition, the housing 710 may be flexible and engaged with the capacitive circuit 708 in this case to assist in the coupling of pressure waves to the capacitive circuit 708. Also, the external surface 722 will form part of the hermetic seal for the device 702 here, along with hermetic sealing (e.g., via adhesive or welding) between the external surface 722 and the hermetic external surface 714 of the housing 710. For example, a thin layer of fiberglass (or some other suitable material) may be provided over an outer enclosure of the capacitive circuit 708 (or directly over an outer plate of the capacitive circuit 708).

In other embodiments, the pressure sensor 704 may be located completely within the housing 710 (e.g., which is biocompatible and provides a hermetic seal). In these cases, the pressure sensor 704 need not be biocompatible or hermetic. However, the housing 710 will be flexible and engaged with the capacitive circuit 708 in this case to efficiently couple pressure waves to the capacitive circuit 708. For example, the housing 710 may comprise a relatively thin outer layer (e.g., constructed of silicone, fiberglass, or some other suitable material) that covers an outer enclosure of the capacitive circuit 108 (or covers an outer plate of the capacitive circuit 108).

FIG. 8 is a simplified perspective view of the pressure sensor device 702 that better illustrates certain three-dimensional aspects of the coil wire of the inductive circuit 706, the plates of the capacitive circuit 708, and the hole 712.

Here, it may be seen that the plates 718A and 718B of the capacitive circuit 708 may take the form of a cylinder or a partial cylinder. Each cylinder is oriented in a longitudinal direction along the longitudinal axis of the housing 710. In a similar manner as described above at FIG. 1, a relatively flexible dielectric material (e.g., a fiberglass material) may be disposed between the plates 718A and 718B. The capacitive circuit 708 may be constructed in various ways (e.g., as described above in conjunction with FIGS. 1, 2, 5, and 6).

The conductor 716 of the inductive circuit 706 may take the form of a cylindrical coil or some other coil-like structure as shown in FIG. 8. The inductive circuit 706 may be constructed in various ways (e.g., as described above in conjunction with FIGS. 1-4).

FIGS. 7 and 8 illustrate an embodiment where the inductive circuit 706 and the capacitive circuit 708 are physically located in a series relationship with respect to one another. In other embodiments (not shown), the capacitive circuit 708 may be located over the inductive circuit 706 in a concentric relationship.

As FIG. 7 illustrates, one terminal of the conductor 716 of the inductive circuit 106 is coupled via a conductor 720A to a plate 718A of the capacitive circuit 708, while another terminal of the conductor 716 is coupled via a conductor 720B to a plate 718B of the capacitive circuit 708. Thus, the inductive circuit 706 and the capacitive circuit 708 are coupled in parallel, thereby forming a passive resonant circuit that is capable of being excited by an externally applied electromagnetic field. As discussed above, the physical properties of the inductive circuit 706 and the capacitive circuit 708 are selected to provide a desired resonant frequency for the sensor circuit 704.

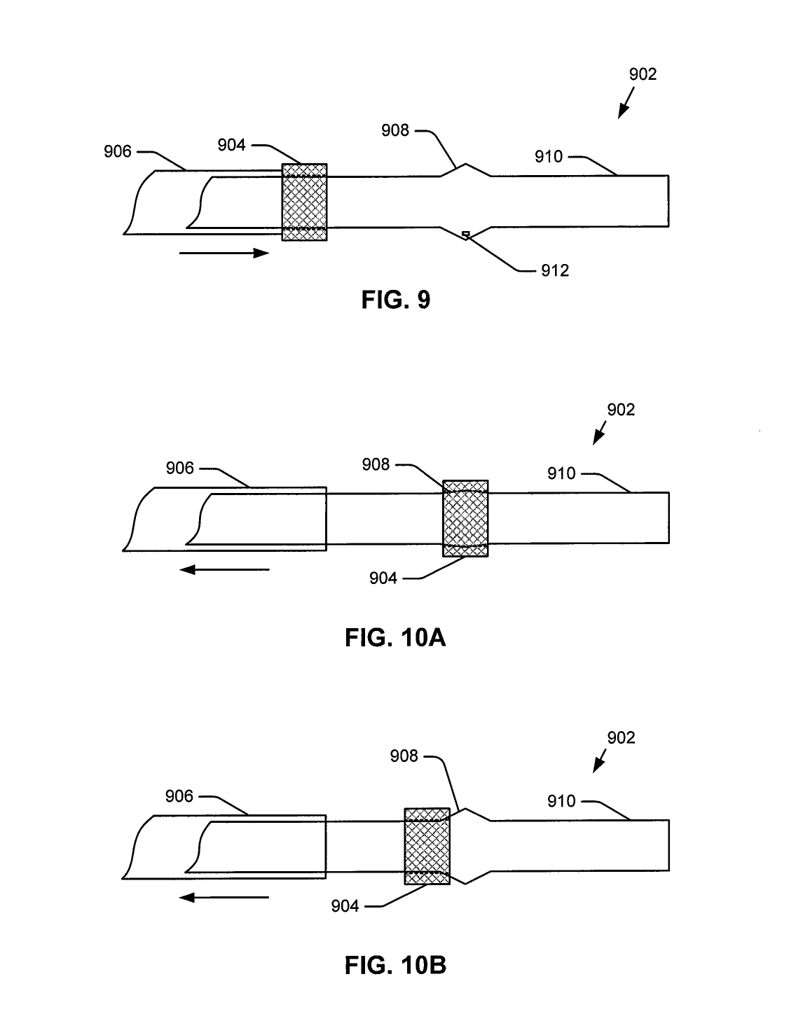

FIGS. 9-12 illustrate, in a simplified manner, two examples of how a pressure sensor device may be installed on an implantable lead. Typically, after a lead is implanted, the pressure sensor device is slid down the lead (from proximal end toward distal end) with a push tool.

Referring initially to FIGS. 9, 10A, and 10B, these figures illustrate an embodiment where a pressure sensor device 904 is mounted over a small protrusion 908 (e.g., a bump) incorporated into or onto the body 910 of an implantable lead 902. FIG. 9 illustrates that a push tool 906 is used to slide the pressure sensor device 904 in a distal direction down the lead body 910. Here, it may be seen that the lead body comprises a small protrusion 908 (e.g., which may be relatively elastic). In some embodiments, the protrusion 908 comprises a radio marker 912 to facilitate identifying the location of the protrusion 908 via x-ray or some other suitable imaging technique.

FIG. 10A illustrates an embodiment where the pressure sensor device 904 is placed over the protrusion 908 (which is elastic in this case). The push tool 906 is removed once the pressure sensor device 904 has been installed over the protrusion 908. In this example, as a result of the protrusion 908 being compressed by the pressure sensor device 904, the protrusion 908 exerts an opposing force against the surface of the hole of the pressure sensor device 904. This force thus tends to secure the pressure sensor device 904 in place on the lead body 910 (i.e., the force tends to prevent the pressure sensor device 904 from moving in either the distal direction or the proximal direction along the lead body 910).

FIG. 10B illustrates an alternate embodiment where the pressure sensor device 904 is positioned up against the protrusion 908 (which may not be elastic in this case). Here, the protrusion 908 serves to prevent the pressure sensor device 904 from travelling further down the lead body 910 (i.e., in the distal direction). In this example, a relatively snug fit between the interior surface of the hole of the pressure sensor device 904 and the outer surface of the lead body may be employed to prevent the pressure sensor device 904 from moving back up the lead body 910 (i.e., in the proximal direction). Again, the push tool 906 is removed once the pressure sensor device 904 has been positioned adjacent the protrusion 908.

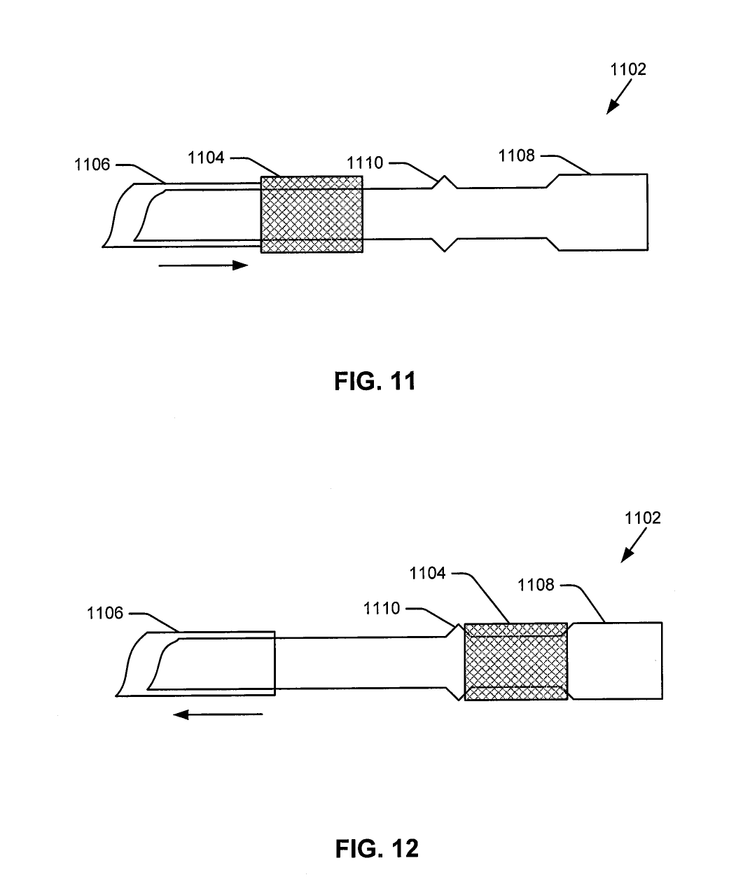

FIGS. 11 and 12 illustrate embodiments where a pressure sensor device 1104 is mounted against a header 1108 and/or a small protrusion 1110 of an implantable lead 1102. FIG. 11 illustrates that a push tool 1106 is used to slide the pressure sensor device 1104 down the implantable lead 1102 in the distal direction. In some embodiments (not shown in FIG. 11 or FIG. 12), the pressure sensor device 1104 is pushed down the implantable lead 1102 and positioned against the protrusion 1110 (e.g., in a similar manner as shown in FIG. 10B). In these embodiments, the protrusion 1110 serves to prevent the pressure sensor device 1104 from travelling further down the implantable lead 1102 (i.e., in the distal direction).

FIG. 12 illustrates an embodiment where the pressure sensor device 1104 is held in place by the header 1108 and the protrusion 1110 (which is elastic in this case). The protrusion 1110 compresses (not shown in FIG. 12) when the sensor device 1104 is pushed over the protrusion 1110. After the sensor device 1104 has passed over the protrusion 1110, the protrusion 1110 returns to its original shape. The size of the header 1108 relative to hole of the pressure sensor device 1104 prevents the pressure sensor device 1104 from sliding any further down the lead 1102 in the distal direction. The protrusion 1110 serves to prevent the pressure sensor device 1104 from sliding back up the lead 1102 in the proximal direction. The push tool 1106 is removed once the pressure sensor device 1104 has been installed in place between the protrusion 1110 and the header 1108. In some implementations, the protrusion 1110 is slightly compressed on its right-hand side by the sensor device 1104. In this case, the protrusion exerts an opposing force that tends to push the pressure sensor device 1104 against the header 1108, thereby firmly holding the pressure sensor device 1104 in place.

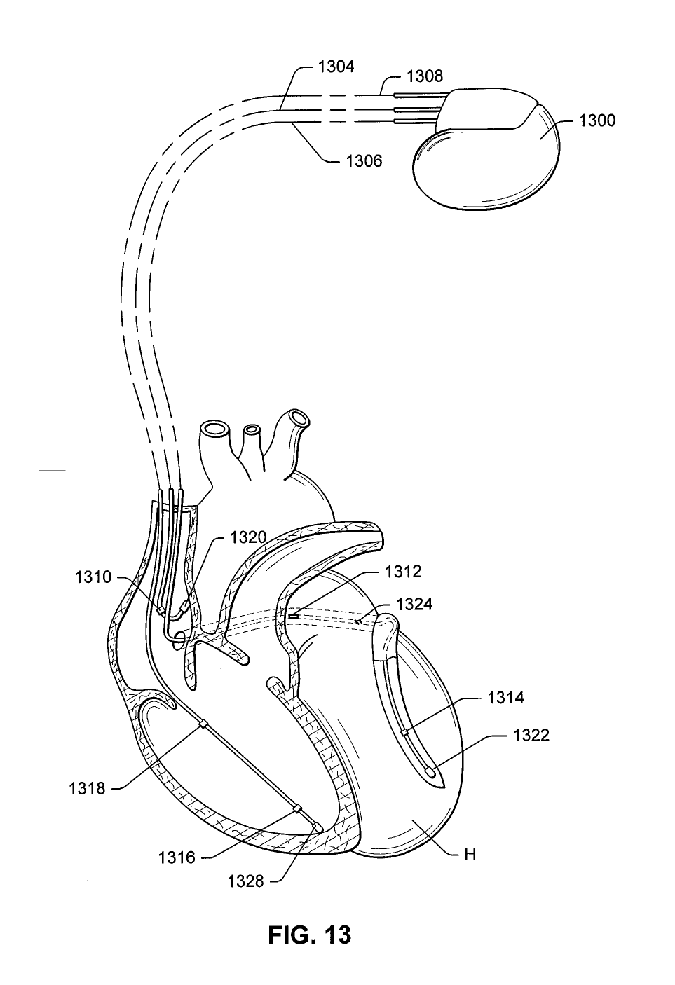

FIG. 13 shows an exemplary implantable cardiac device 1300 in electrical communication with a patient's heart H by way of three leads 1304, 1306, and 1308, suitable for delivering multi-chamber stimulation and shock therapy. Bodies of the leads 1304, 1306, and 1308 (or another other leads describe herein) may be formed of silicone, polyurethane, plastic, or similar biocompatible materials to facilitate implant within a patient. Each lead includes one or more conductors, each of which may couple one or more electrodes incorporated into the lead to a connector on the proximal end of the lead. Each connector, in turn, is configured to couple with a complimentary connector (e.g., implemented within a header) of the device 1300.

To sense atrial cardiac signals and to provide right atrial chamber stimulation therapy, the device 1300 is coupled to an implantable right atrial lead 1304 having, for example, an atrial tip electrode 1320, which typically is implanted in the patient's right atrial appendage or septum.

To sense left atrial and ventricular cardiac signals and to provide left chamber pacing therapy, the device 1300 is coupled to a coronary sinus lead 1306 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 1306 is designed to receive atrial and ventricular cardiac signals and to deliver left ventricular pacing therapy using, for example, a left ventricular tip electrode 1322; and provide left atrial pacing therapy using, for example, a left atrial ring electrode 1324. For a more detailed description of a coronary sinus lead, the reader is directed to U.S. Pat. No. 5,466,254, “Coronary Sinus Lead with Atrial Sensing Capability” (Helland), which is incorporated herein by reference.

The device 1300 is also shown in electrical communication with the patient's heart H by way of an implantable right ventricular lead 1308 having, in this implementation, a right ventricular tip electrode 1328. Typically, the right ventricular lead 1308 is transvenously inserted into the heart H to place the right ventricular tip electrode 1328 in the right ventricular apex. Accordingly, the right ventricular lead 1308 is capable of sensing or receiving cardiac signals, and delivering stimulation in the form of pacing to the right ventricle.

It should be appreciated that the device 1300 may connect to leads other than those specifically shown. In addition, the leads connected to the device 1300 may include components other than those specifically shown. For example, a lead may include other types of electrodes, shocking coils, sensors or devices that serve to otherwise interact with a patient or the surroundings.

In accordance with the teachings herein, one or more of the leads 1304, 1306, or 1308 may incorporate a passive pressure sensor. For example, the lead 1304 may incorporate (e.g., integrated as in FIG. 1 or attached as in FIG. 10) a passive pressure sensor 1310 in some embodiments. Similarly, in various embodiments, the lead 1306 may incorporate (e.g., integrated as in FIG. 1 or attached as in FIG. 10) a passive pressure sensor 1312 and/or a passive pressure sensor 1314. Also, in various embodiments, the lead 1308 may incorporate (e.g., integrated as in FIG. 1 or attached as in FIG. 10) a passive pressure sensor 1316 and/or a passive pressure sensor 1318.

As discussed above, an implantable lead may be oriented to place a pressure sensor at a desired location within the heart. For example, to obtain a relatively accurate estimate of pulmonary artery pressure using an RV lead, it may be preferable to place the RV lead in the outflow tract of the heart. Thus, an RV lead may be preformed into a J-shape to facilitate placing a pressure sensor on the RV lead at the outflow tract.

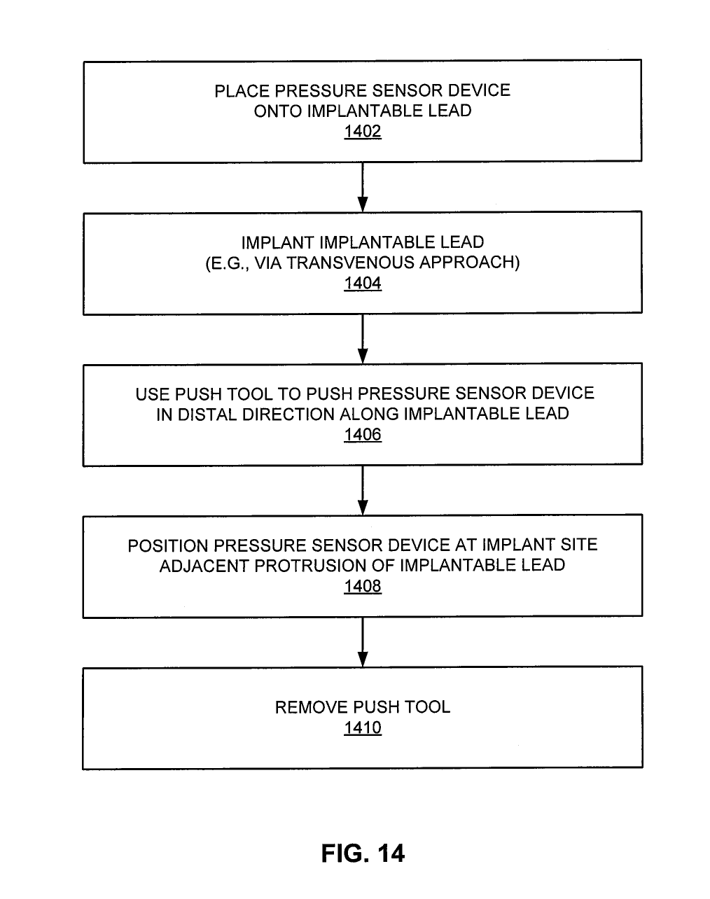

FIG. 14 illustrates sample operations that may be performed to install a pressure sensor device over an implantable lead). As discussed herein, the pressure sensor device (e.g., the pressure sensor device 702) may have a substantially hollow cylinder shape defining a hole, wherein the hole has a diameter that is slightly larger than an outer diameter of an implantable lead

For convenience, the operations of FIG. 14 (or any other operations discussed or taught herein) may be described as being performed by specific components. It should be appreciated, however, that these operations may be performed by other types of components and may be performed using a different number of components. It also should be appreciated that one or more of the operations described herein may not be employed in a given implementation.

As represented by block 1402, the pressure sensor device is placed onto an implantable lead (e.g., at or near a proximal end of the implantable lead). This operation may be performed at different times in different implementations. In some implementations, this operation is performed when the implantable lead is built. For example, the pressure sensor device may be slid onto the lead body before connectors or other end components are incorporated into the implantable lead. In other implementations, the pressure sensor device placement operation is performed during the lead implant procedure.

As represented by block 1404, the implantable lead is implanted in a patient. For example, an RV lead may be implanted via a transvenous approach whereby the distal end of the RV lead is ultimately positioned in the RV of the patient's heart. The implantable lead may then be fixed in place by an appropriate fixation technique (e.g., active and/or passive). At this point, the pressure sensor device will have been placed on the implantable lead at a proximal section of the implantable lead (e.g., during manufacture or during the implant procedure).

As represented by block 1406, a push tool (e.g., a sheath, a wire, or some other suitable structure) is used to push the pressure sensor device in the distal direction along the implantable lead. As represented by block 1408, the pressure sensor device is pushed down the implantable lead until it is positioned at the desired implant site adjacent (e.g., over or next to) a protrusion (e.g., a bumper, a header, etc.) of the implantable lead. As discussed herein, the protrusion may be formed as part of the implantable lead, attached to the implantable lead, or incorporated into the implantable lead in some other suitable manner. In some embodiments, the protrusion is elastic and compressible. In some embodiments, the protrusion protrudes from an exterior surface of the implantable lead (e.g., from the lead body) by a distance that is less than or equal to 2 mils.

As represented by block 1410, once the pressure sensor device is implanted at the desired location, the push tool is removed. A suitable external monitoring device may then be used (e.g., on a daily basis) to measure pressure at the implant site by generating an electromagnetic field that excites the resonant circuit of the pressure sensor device, and then monitoring changes in the resulting resonant frequency.

It should be appreciated from the above that the various structures and functions described herein may be incorporated into a variety of apparatuses (e.g., a sensing lead, a pacing lead, a monitoring device, etc.) and implemented in a variety of ways. Different embodiments of such an apparatus may include a variety of hardware and software processing components. In some embodiments, hardware components such as processors, controllers, state machines, logic, or some combination of these components, may be used to implement some of the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in any methods (e.g., processes) disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements. In addition, terminology of the form “at least one of A, B, or C” or “one or more of A, B, or C” or “at least one of the group consisting of A, B, and C” used in the description or the claims means “A or B or C or any combination of these elements.”

As used herein, the term “determining” encompasses a wide variety of actions. For example, “determining” may include calculating, computing, processing, deriving, investigating, looking up (e.g., looking up in a table, a database or another data structure), ascertaining, and the like. Also, “determining” may include receiving (e.g., receiving information), accessing (e.g., accessing data in a memory), and the like. Also, “determining” may include resolving, selecting, choosing, establishing, and the like.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure.

What is claimed is:

1. An implantable lead, comprising:
a biocompatible lead body;
at least one electrical circuit;
a inductor-capacitor resonant circuit that is electrically isolated from the at least one electrical circuit and any other electrical circuit of the implantable lead, wherein the inductor-capacitor resonant circuit comprises an inductive circuit and a flexible capacitive circuit electrically coupled in parallel; and
a flexible insulator material located adjacent an exterior surface of the lead body and engaged with the capacitive circuit to couple pressure waves to the capacitive circuit.

2. The implantable lead of claim 1, wherein the capacitive circuit comprises a plurality of concentric partially-cylindrical plates oriented in a longitudinal direction along a longitudinal axis of the lead body.

3. The implantable lead of claim 1, wherein the capacitive circuit comprises a plurality of concentric cylindrical plates oriented in a longitudinal direction along a longitudinal axis of the lead body.

4. The implantable lead of claim 1, wherein the inductor-capacitor resonant circuit has a resonant frequency of 35 MHz or less.

5. The implantable lead of claim 1, wherein the inductor-capacitor resonant circuit has a resonant frequency of 20 MHz or less.

6. The implantable lead of claim 1, wherein the at least one electrical circuit comprises at least one electrode.

7. The implantable lead of claim 6, further comprising a connector coupled to a proximal end of the lead body and wherein the at least one electrical circuit further comprises at least one conductor coupled to the at least one electrode and the connector.

8. A pressure sensor device for an implantable lead, comprising:
a flexible biocompatible housing defining a central hole along a longitudinal axis of the biocompatible housing, wherein the housing has an outer diameter of less than 3 millimeters; and
an inductor-capacitor resonant circuit embedded within the housing, wherein the inductor-capacitor resonant circuit comprises an inductive circuit and a flexible capacitive circuit electrically coupled in parallel, and wherein the housing is engaged with the capacitive circuit to couple pressure waves to the capacitive circuit.

9. The pressure sensor device of claim 8, wherein the central hole has a diameter of less than 2.5 millimeters.

10. The pressure sensor device of claim 8, wherein the housing has a length of less than 80 millimeters.

11. The pressure sensor device of claim 8, wherein the capacitive circuit comprises a plurality of concentric partially-cylindrical plates oriented in a longitudinal direction along a longitudinal axis of the housing.

12. The pressure sensor device of claim 8, wherein the capacitive circuit comprises a plurality of concentric cylindrical plates oriented in a longitudinal direction along a longitudinal axis of the housing.

13. The pressure sensor device of claim 8, wherein the inductor-capacitor resonant circuit has a resonant frequency of 35 MHz or less.

14. The pressure sensor device of claim 8, wherein the inductor-capacitor resonant circuit has a resonant frequency of 20 MHz or less.

15. The pressure sensor device of claim 8, wherein the housing has an outer diameter of greater than 2 millimeters.

16. The pressure sensor device of claim 8, wherein:
the central hole has a diameter of less than 2.5 millimeters;
the housing has an outer diameter of greater than 2 millimeters;
the housing has a length of less than 80 millimeters; and
the capacitive circuit comprises a plurality of concentric semi-cylindrical plates oriented in a longitudinal direction along a longitudinal axis of the housing.

17. An implant method for a pressure sensor device that has a substantially hollow cylinder shape defining a hole, wherein the hole has a diameter that is slightly larger than an outer diameter of an implantable lead, the method comprising:
implanting a distal section of the implantable lead within a heart of a patient via a transvenous approach, wherein the pressure sensor device is placed on the implantable lead at a proximal section of the implantable lead;
pushing the pressure sensor device in a distal direction along the implanted lead; and
positioning the pressure sensor device adjacent a protrusion of the implantable lead.

18. The method of claim 17, wherein the pressure sensor device comprises:
a flexible biocompatible housing defining a central hole along a longitudinal axis of the biocompatible housing, wherein the housing has an outer diameter of less than 3 millimeters; and
an inductor-capacitor resonant circuit embedded within the housing, wherein the inductor-capacitor resonant circuit comprises an inductive circuit and a flexible capacitive circuit electrically coupled in parallel, and wherein the housing is engaged with the capacitive circuit to couple pressure waves to the capacitive circuit.

19. The method of claim 18, wherein:
the central hole has a diameter of less than 2.5 millimeters;
the housing has an outer diameter of greater than 2 millimeters;
the housing has a length of less than 80 millimeters.

20. An implantable lead, comprising:
a lead body comprising a biocompatible material, wherein the lead body comprises at least one protrusion that protrudes from an outer surface of the lead body;
at least one electrode incorporated within the lead body; and
at least one conductor incorporated within the lead body and coupled to the at least one electrode.

21. The implantable lead of claim 20, wherein the protrusion is elastic and compressible.

22. The implantable lead of claim 20, wherein the protrusion protrudes from the outer surface by less than 20 mils.
